(19) United States
(12) Patent Application Publication
(10) Pub. No.: US 2004/0009156 A1
(43) Pub. Date: Jan. 15, 2004

(54) Antisense therapy using oligonucleotides that target human kinesin genes for treatment of cancer

(76) Inventors: Christoph Reinhard, Alameda, CA (US); Annette Walter, San Carlos, CA (US)

Correspondence Address: Steven W. Collier, Chiron Corporation, 4560 Horton Street, Emeryville, CA, 94608-2916, US

(21) Appl. No.: 10/269,021

(22) Filed: Oct. 10, 2002

Related U.S. Application Data
(60) Provisional application No. 60/328,444, filed on Oct. 12, 2001.

(51) Int. Cl. A61K 48/00

(52) U.S. Cl. 424/93.21; 514/44

(57) ABSTRACT

The present invention is directed toward the use of antisense oligonucleotides that target human kinesin genes for treating diseases involving aberrant cell proliferation, particularly cancers such as colon cancer. Also, the invention is directed to a synergistic combination for treating cancer comprising a chemotherapeutic such as cisplatin and an antisense oligonucleotide that specifically inhibits human kinesin expression.

### BACKGROUND OF THE INVENTION

1. Field of the Invention

The present invention provides novel therapeutics for treatment and/or prevention of cancers and other cell disorders involving aberrant cell proliferation that target human kinesin genes. More specifically, the present invention provides antisense oligonucleotides that inhibit the expression of human kinesin genes and compositions containing. These antisense oligonucleotides and compositions containing are useful especially for treating and/or prophylaxis of colon cancer. These antisense oligonucleotides are useful alone or in combination, particularly with other therapeutics, e.g. chemotherapeutics such as cisplatin.

2. Description of Related Art

Translocation of components within the cell is critical for maintaining cell structure and function. Cellular components such as proteins and membrane-bound organelles are transported along well-defined routes to specific subcellular compartments. Intracellular transport mechanisms utilize microtubules which are filamentous polymers that serve as tracks for directing the movement of molecules. Molecular transport is driven by the microtubule-based motor proteins, kinesin and dynein. These proteins use the energy derived from ATP hydrolysis to power their movement unidirectionally along microtubules and to transport molecular cargo to specific destinations.

Kinesin defines a ubiquitous, conserved family of over 50 proteins that can be classified into at least eight subfamilies based on primary amino acid sequence, domain structure, velocity of movement and cellular function [Reviewed in Moore, J. D. and Endow, S. A. (1996) Bioessays 18:207-219; and Hoyt, A. M. (1994) Curr. Opin. Cell Biol. 6:63-68]. The prototypical kinesin molecule is involved in the transport of membrane-bound vesicles and organelles. This function is particularly important for axonal transport in neurons. Protein-containing vesicles are constantly transported from the neuronal cell body along microtubules that span the length of the axon leading to the synaptic terminal. Failure to supply the synaptic terminal with these vesicles blocks the transmission of neural signals. In the fruit fly Drosphila melanogaster, for example, mutations in kinesin cause severe disruption of axonal transport in larval nerves which lead to progressive paralysis [Hurd, D. D. and Saxton, W. M. (1996) Genetics 144:1075-1085]. This phenotype mimics the pathology of some vertebrate motor neuron diseases, such as amyotrophic lateral sclerosis (ALS). In addition to axonal transport, kinesin is also important in all cell types for the transport of vesicles from the Golgi complex to the endoplasmic reticulum. This role is critical for maintaining the identity and functionality of these secretory organelles.

Members of the more divergent subfamilies of kinesin are called kinesin-related proteins (KRPs), many of which function during mitosis in eukaryotes as divergent as yeast and human (Hoyt, supra). Some KRPs are required for assembly of the mitotic spindle. In vivo and in vitro analyses suggest that these KRPs exert force on microtubules that comprise the mitotic spindle, resulting in the separation of spindle poles. Phosphorylation of KRP is required for this activity. Failure to assemble the mitotic spindle results in abortive mitosis and chromosomal aneuploidy, the latter condition being characteristic of cancer cells. In addition, a unique KRP, centromere protein E, localizes to the kinetochore of human mitotic chromosomes and may play a role in their segregation to opposite spindle poles.

The prototypical kinesin molecule is a heterotetramer comprised of two heavy polypeptide chains (KHCs) and two light polypeptide chains (KLCs). The KHC subunits are typically referred to as “kinesin”. KHC is about 1000 amino acids in length, and KLC is about 550 amino acids in length. Two KHCs dimerize to form a rod-shaped molecule with three distinct regions of secondary structure. At one end of the molecule is a globular motor domain that functions in ATP hydrolysis and microtubule binding. Kinesin motor domains are highly conserved and share over 70% identity. Beyond the motor domain is an α-helical coiled-coil region which mediates dimerization. At the other end of the molecule is a fan-shaped tail that associates with molecular cargo. The tail is formed by the interaction of the KHC C-termini with the two KLCs.

The nematode Unc-104 kinesin-like protein defines a distinct kinesin subfamily whose members may function monomerically (Moore and Endow, supra). Members of this subfamily are important for synaptic transport and mitochrondial translocation and are characterized by movement at relatively high velocities of about 40 to 90 microns/minute. Nematodes with mutations in the Unc-104 gene exhibit defects in locomotion and feeding behaviors, and at the molecular level, in synaptic vesicle transport.

It has further been reported that certain kinesins are associated with neoplastic transformation and the sensitivity of cells to certain drugs (see U.S. Pat. No. 6,043,340 by Gudkov et al., U.S. Pat. No. 5,665,550 by Robinson et al., U.S. Pat. No. 5,942,384 by Kirchling et al., U.S. Pat. No. 5,866,327 by Gudkov et al. and U.S. Pat. No. 6,083,746 by Gudkov et al.). These patents disclose in particular the selection of genetic suppressor elements, including antisense oligonucleotides that impart resistance to chemotherapeutics such as cisplatin, or which confer a transformed phenotype to untransformed cells which are derived from a kinesin gene.

It has also been reported that expression of an antisense RNA fragment derived from mouse ubiquitous kinesin heavy chain (uKHC) cDNA is associated with a unique type of multidrug resistance [Axenovich et al., Cancer Res. 58:3423-8 (1998)]. See also Efferty, et al., Anticancer Res. 20:3211-9(2000) who teaches that this gene is involved in drug resistance.

Also, it has been reported that there is increased chromokinesin in retinolastoma cells and that this observation may be useful in designing strategies to modulate cell proliferation and cell movement, e.g. by use of antibodies or antisense oligos.

Further, it has been reported that a particular kinesin gene, HK2, is expressed at higher levels in tumor cells after treatment with a synthetic retinoid N-(4-hydrotymethyl)-cell-trans-retinamide (HPR), a cancer chemopreventive and inducer of apoptosis [Debernardi et al., Genomics 42(1):67-73 (1997)].

Still further, it has been reported that kinesin expression may be associated with the invasive and metastatic phenotypes of human prostate tumor sublines [Steams et al., Cancer Res. 51:5866-75 (1991)].

### OBJECTS OF THE INVENTION

It is an object of the invention to provide antisense oligonucleotides that inhibit the expression of human kinesin genes.

It is another object of the invention to provide pharmaceutical compositions for the treatment of cancer and other disorders that involve aberrant cell proliferation by administration of antisense oligonucleotides which inhibit the expression of human kinesin genes.

It is another object of the invention to provide methods of treating cancer or other disorders involving aberrant cell proliferation by the administration of antisense oligonucleotides that inhibit the expression of human kinesin genes.

It is a more specific object of the invention to provide methods for treating colon cancer that comprise the administration of at least one antisense oligonucleotide that inhibits the expression of human kinesin genes.

It is another specific object of the invention to render cells more susceptible to chemotherapy by administering a combination of a chemotherapeutic and an antisense oligonucleotide that inhibits human kinesin expression, wherein said chemotherapeutic and antisense oligonucleotide may be administered together or separately, and in either order.

It is an even more specific object of the invention to render cells more susceptible to cisplatin by administering a combination of cisplatin and an antisense oligonucleotide that inhibits human kinesin gene expression, wherein said cisplatin and oligonucleotide are administered together or separately, and in either order.

### BRIEF DESCRIPTION OF THE INVENTION

The invention is based on the discovery that antisense oligonucleotides may be designed that target specific human kinesin and kinesin-like molecules which are useful for treating cancer and other disorders involving aberrant cell proliferation.

In particular, antisense oligonucleotides were designed based on the structure of specific human kinesin genes which reduce or inhibit the expression of one or more kinesin gene members. The efficacy of the subject antisense oligonucleotides to inhibit the expression of targeted kinesin genes was established by use of real time PCR analyses.

It was further shown that when these antisense oligonucleotides were transfected into a human colon cancer cell line SW620 that they significantly (relative to an appropriate reverse control) inhibited the ability of such cells to grow in soft agar, an accepted assay for measuring anchorage independent growth, a hallmark of tumorigenesis.

Still further, it was shown that transient transfection of SW620 cells with antisense oligonucleotides according to the invention arrested cells in G2/M phase to various degrees.

Also, it was demonstrated that antisense oligonucleotides according to the invention potentiate the efficacy of chemotherapeutics, particularly cisplatin.

Based on these observations, it is anticipated that the present oligonucleotides may be used to treat cancer and other disorders involving aberrant cell proliferation.

### BRIEF DESCRIPTION OF THE DRAWINGS

FIG. 1 shows the effect of different kinesin anti-sense oligonucleotides and reverse controls on anchorage independent growth.

FIG. 2 shows the effect of kinesin anti-sense oligonucleotides and corresponding reverse controls on the cell cycle profile of SW620 cells.

FIG. 3 shows the effect of several kinesin antisense oligonucleotides and reverse controls on the cell cycle profile of normal human fibroblasts.

FIG. 4 shows the effect of different kinesin antisense oligonucleotides, corresponding reverse controls and chemotherapy (cisplatin) on cytotoxicity of SW620 cells.

FIG. 5 shows the effect of different antisense oligonucleotides, corresponding reverse controls, and cisplatin on MRC9 cells.

FIG. 6 shows the effect of several kinesin antisense oligonucleotides and corresponding reverse controls on the cell proliferation of SW620 cells.

FIG. 7 shows the effect of several kinesin antisense oligonucleotides and corresponding reverse controls on the proliferation of normal cells.

### PREFERRED EMBODIMENTS OF THE INVENTION

A method of treating and/or preventing a disease involving aberrant cell proliferation comprising administering a therapeutically or prophylactically effective amount of an antisense oligonucleotide that inhibits the expression of a kinesin gene.

A method of treating and/or preventing a disease involving aberrant cell proliferation comprising administering at least one antisense oligonucleotide that inhibits or reduces the expression of a kinesin gene, wherein said anti-sense oligos selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

A method of treating and/or preventing cancer comprising administering a therapeutically or prophylactically effective amount of at least one antisense oligonucleotide that specifically inhibits the expression of a human kinesin gene.

A method of treating and/or preventing cancer comprising administering at least one antisense oligonucleotide that inhibits or reduces the expression of a kinesin gene selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

A method of enhancing the efficacy of a chemotherapeutic comprising administering said chemotherapeutic with at least one antisense oligonucleotide that specifically inhibits the expression of a human kinesin gene.

A method of treating and/or preventing colon cancer comprising administering to a patient in need of such treatment or prevention a therapeutically or prophylactically effective amount of at least one antisense oligonucleotide selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

A composition containing an antisense oligonucleotide selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

An antisense oligonucleotide selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

A method of enhancing the efficacy and/or reducing the required therapeutic dosage of a chemotherapeutic agent comprising administering said chemotherapeutic in combination with at least one antisense oligonucleotide selected from the group consisting of:

| | |
|---|---|
| CCTCCGCCATCCTATCAGGCTGAA | (SEQ ID NO:1) |
| CCGAGGAGAAAGCGAAATAGGGAAG | (SEQ ID NO:2) |
| GAGACCGACTCTTGCTCTGTTGCC | (SEQ ID NO:3) |
| GTTGATCTGGGCTCGCAGAGGTAAT | (SEQ ID NO:4) |
| CTCTGTGGTGTCGTACCTGTTGGGA | (SEQ ID NO:5) |
| TGGGTTCAAGTGATTCTCGTGCCTC | (SEQ ID NO:6) |
| TGTCAGCCAATCCTCCAGTTCGTAC | (SEQ ID NO:7) |
| TTGTACGCCCTCCAAGAGAATCCTG | (SEQ ID NO:8) |
| GCTCAAGCAATCCACCCGCCTCAG | (SEQ ID NO:9) |
| GGGATTACAGGCATGAGCCACCGC | (SEQ ID NO:10) |
| CACTCCATTTTTCTCACGGGCTGCA | (SEQ ID NO:11) |
| CATTCTCCTGAGCCGTGATGCGAA | (SEQ ID NO:12) |
| ACGGAACGGGGTGTGAGCCTTGT | (SEQ ID NO:13) |
| TGTCAGCTTGCTCTCACGGAACGG | (SEQ ID NO:14) |
| GGAGCTTATGCCTGGTGAGATCGTG | (SEQ ID NO:15) |
| GAGTCAGCAAGGAAGAGAAACGCG | (SEQ ID NO:16) |
| TGGATAAATTGCCTGGAATCAGCGC and | (SEQ ID NO:17) |
| CGTTGGATCTTGATAGCGAGACCGG. | (SEQ ID NO:18) |

wherein said chemotherapeutic and antisense oligonucleotide are administered separately or in combination and in either order.

### DETAILED DESCRIPTION OF THE INVENTION

The present invention employs oligomeric antisense oligonucleotides, for use in modulating the function of nucleic acid molecules encoding human kinesin, ultimately modulating the amount of human kinesin produced. This is accomplished by providing antisense compounds which specifically hybridize with one or more nucleic acids encoding human kinesin. As used herein, the terms “target nucleic acid” and “nucleic acid encoding human kinesin” encompass DNA encoding human kinesin, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of human kinesin. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense therapy. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding human kinesin. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding human kinesin, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the genet and the 3′ untranslated region (3′UTR), known in the art to refer to the portion of an mRNA in the 3′ direction from tie translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via 4′-5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or, where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e, hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to nontarget sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleosides).

Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or no natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar backbone of an oligonucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

Other preferred modifications include 2′-methoxy (2′-OCH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are presently preferred base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-S-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNAduplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have alto been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5;403,711; 5,491,133; 5,565,350; 5,623,065; 5,652,355; 5,652,356; and 5,700,922, certain of which are commonly owned with the instant application, and each of which is herein incorporated by reference in its entirety.

The antisense compounds used in accordance with this invention may be conveniently and routinely made through the well-known techniques of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative U.S. patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5;547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,169,854; 5,512,295; 5,527,528; 5,534,259; 5,543,152; 5,56,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2thioethyl) phosphates derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as canons are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma. Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of a human kinesin gene treated by administering antisense compounds in accordance with this invention. The compounds of the invention can be utilized in pharmaceutical compositions by adding an effective amount of an antisense compound to a suitable pharmaceutically-acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding human kinesin, enabling sandwich and other assays to easily be constructed to exploit this fact. Hybridization of the antisense oligonucleotides of the invention with a nucleic acid encoding human kinesin can be detected by means known in the art. Such means may include conjugation of an enzyme to the oligonucleotide, radiolabelling of the oligonucleotide or and other suitable detection means. Kits using such detection means for detecting the level to human kinesin in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery) pulmonary, e.g., by inhalation or insufflation of powders or aerosols, including by nebulizer; intratracheal, intranasal epidermal and transdermal), oral or parenteral. Parentera: administration includes intravenous, intraarterial, subcutaneous, intraperitoneal or intramuscular injection or infusion; or intracranial, e,g., intrathecal or intraventricular administration. Oligonucleotides with at least one 2′-O methoxyethyl modification are believed to be particularly useful for oral administration.

Pharmaceutical compositions and formulations for topical administration may include transdermal patches, ointments, lotions, creams, gels, drops, suppositories, sprays, liquid; and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions it water or non-aqueous media, capsules, sachets or tablets, thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solid: and self-emulsifying semisolids.

The pharmaceutical formulations of the present invention which may conveniently be presented in unit dosage form may be prepared according to conventional techniques well-known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marvel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic, and amphoteric (Rieger, in Pharmaceutical Dosage Forms, Lieberman, Rieger and Banker (Eds.), 1988, Marcel Dekker, Inc., New York, N.Y., volume I, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.),1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water insoluble drugs in a formulation of thermodynamically stable droplets that are formed spontaneously.

Surfactants used in the preparation of microemulsions include, but are not limited to, ionic surfactants, non-ionic surfactants, Brij 96, polyopcyethylene oleyl ethers, polyglycerol fatty acid esters, tetcaglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (M0750), docaglycerol sequioleate (S0750), decaglycerol decaoleate (IbA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five bread categories-surfactants, fatty acids, bile salts, chelating agents, and non-chelating non-surfactants (Lee et al., Critical Reviews in Therapeutic Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.,

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can he formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al., S. TP. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G, N, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. NY Acad. Sci., 1987 507, 64) reported the ability of monosialoganglioside Gm, galactocerebroside sulfate and phosphatidylinositol to improve blood half-lives of liposomes. These findings were expounded upon by Gabizon et al. (Proc. Natl. Acad. Sci. U.SA., 1988, 85, 6949). U.S. Pat. No. 4,837,028 and WO 88/04924, both to Allen et al., disclose liposomes comprising (1) sphingomyelin and (2) the ganglioside Gm, or a galactocerebroside sulfate ester. U.S. Pat. No. 5,543,152 (Webb et al.) discloses liposomes comprising sphingomyelin. Liposomes comprising 1,2-sndimyristoylphosphatidyl choline are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.). Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers: In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-racglycerol), dilaurin, caprylic acid, arachidonic acid, glycerol 1-monocaprate, 1-dodecylazacycloheptan-2-one, acylcamitines, acylcholines, C1-C10 alkyl esters thereof (e.g., methyl, isopropyl and t-butyl), and mono- and di-glycerides thereof (i.e., oleate, laurate, caprate, myristate, palpitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fatsoluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium tau rodeoxycholate), chenodeoxycholic acid (sodium chenodeoxycholate), ursodeoxycholic acid (UDCA), sodium tauro-24, 25-dihydro-fusidate (STDHF), sodium glycodihydrofusidate and polyoxyethylene-9-lauryl ether (POE) (Lee et al., Critical Reviews in Therapeutic Drug Carrier Systems, 1991, p. 92; Swinyard, Chapter 39 In Remington's Pharmaceutical Sciences, 18th Ed., Gennaro, ed., Mack Publishing Co., Easton, Pa., 1990, pp. 782-783; Muranishi, Critical Reviews in Therapeutic Drug Carrier Systems, 1990, 7:1-33; Yamamoto et al., J. Pharm. Exp. Ther., 1992, 263:25; Yamashita et al., J. Pharm. Sci., 1990, 79:579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618:315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7:1-33; Buur et al. J. Control Rel., 1990, 14:43-51).

Non-chelating non-surfactants: As used herein, nonchelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7:1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39:621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5:115-121; Takakura et al., Antisense & Nucl. Acid Drug Dev, 1996, 6:177-183).

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, nonaqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine 6-thioguanine, cytarabine (CA), 5-fluorouracil (5-FU), floxuridine (5-FUdR), methotrexate (MTX), colehicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on ECsos found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

As noted, the present invention relates to the use of antisense oligos that target human kinesin genes for treating disorders involving aberrant cell proliferation. Examples thereof include especially cancers, autoimmune disorders, viral infections, neurological disorders, conditions associated with ischemia such as myocardial infarction and strokes.

Examples of cancers treatable according to the invention include colon cancer, breast cancer, T and B cell lymphomas, leukemias, bladder cancer, pancreatic cancer, stomach cancer, brain cancer, esophageal cancer, liver cancer, adrenalcarcinoma, lung cancer, testicular cancer, ovarian cancer, uterine cancer, head and neck cancer, bone cancer, cervical cancer, heart cancer, gall bladder cancer, parathyroid cancer, penile cancer, prostate cancer, skin cancer, spleen cancer, thymus cancer, thyroid cancer, muscle cancer, ganglial cancer, melanoma, myeloma, sarcoma, and teratocarcinoma, among others.

Preferred cancers for treatment according to the invention are colon cancer, lymphomas and pancreatic cancer.

Examples of neurological disorders that may be treated using kinesin targeted antisense oligos include disorders such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and raduclitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangiolastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety and schizophrenic disorders, akathesia, amnesia, catatonia, diabetic neuropathy, tardive dyskinesia, dystonias, paranoid psychoses, postherpetic neuralgia and Tourette's disorder; and disorders of vesicular transport such as cystic fibrosis, glucose-galactose malabsorption syndrome, hypercholesterolemia, diabetes mellitus, diabetes insipidus, hyper- and hypoglycemia, Grave's disease, goiter, Cushing's disease, Addison's disease, gastrointestinal disorders including ulcerative colitis, gastric and duodenal ulcers, other conditions associated with abnormal vesicle trafficking including acquired immunodeficiency syndrome (AIDS), allergic reactions, autoimmune hemolytic anemia, proliferative glomerulonephritis, inflammatory bowel disease, multiple sclerosis, myasthenia gravis, rheumatoid arthritis, osteoarthritis, scleroderma, Chediak-Higashi syndrome, Sjogren's syndrome, systemic lupus erythematosus, toxic shock syndrome, traumatic tissue damage and viral, bacterial, fungal, helminthic and protozoal infections.

Also, aberrant cell proliferation is associated with a number of liver disorders including obstructive jaundice and hepatic damage due to toxins and drugs, as well as kidney diseases, such as polycistic kidney, and some pancreatic disorders, including diabetes.

As noted above, a preferred embodiment of the invention to administer the subject antisense oligos in combination with other therapeutics as synergistic results may be obtained. Particularly, the combined use of antisense oligos and chemotherapy or radiotherapy for treatment of cancers is contemplated as it has been shown that antisense oligos according to the invention may decrease the requisite therapeutic dosage of some chemotherapeutics. Radionuclides useful in the invention include by way of example ⁹⁰Y, ¹³¹I, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi.

Chemotherapeutics useful according to the invention include cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. An especially preferred chemotherapeutic is cisplatin.

Other suitable cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic.

The chemotherapeutic agent may alternatively comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Other cytotoxins, useful in the invention include ricin subunit A, abrin, diptheria toxin, botulinum, cyanginosins, saxitoxin, shigatoxin, tetanus, tetrodotoxin, trichothecene, verrucologen or a toxic enzyme.

Also, the use of other agents with the subject antisense compounds is contemplated, e.g. cytokines (such as interferons, colony stimulating factors, tumor necrosis factors, interleukins), drugs that inhibit angiogenesis, therapeutic antibodies and other therapeutics suitable for treating disorders involving aberrant cell proliferation such as cancer.

While the present invention has been described with specificity in accordance with certain of its preferred embodiments, the following examples serve only to illustrate the invention and are not intended to limit the same.

### EXAMPLES

### Example 1

As set forth below, Kinesin antisense oligos were designed based on reported human kinesin sequences, i.e. CENP-E (GenBank ID Z15005), human Eg5 (also known as hsKSP or KNSL1) (GenBank ID U37426) and MCAK (also known as KNSL6) (GenBank ID U63743).

More particularly, the following antisense oligonucleotides were synthesized based on the sequence of human kinesin genes as well as the corresponding reverse control sequences:

| | |
|---|---|
| CCTCCGCCATCCTATCAGGCTGAA | (SEQ ID NO:1) |
| AAGTCGGACTATCCTACCGCCTCC | (SEQ ID NO:19) |
| CCGAGGAGAAAGCGAAATAGGGAAG | (SEQ ID NO:2) |
| GAAGGGATAAAGCGAAAGAGGAGCC | (SEQ ID NO:20) |
| GAGACCGACTCTTGCTCTGTTGCC | (SEQ ID NO:3) |
| CCGTTGTCTCGTTCTGAGCCAGAG | (SEQ ID NO:21) |
| GTTGATCTGGGCTCGCAGAGGTAAT | (SEQ ID NO:4) |
| TAATGGAGACGCTCGGGTCTAGTTG | (SEQ ID NO:22) |
| CTCTGTGGTGTCGTACCTGTTGGGA | (SEQ ID NO:5) |
| AGGGTTGTCCATGCTGTGGTGTCTC | (SEQ ID NO:23) |
| TGGGTTCAAGTGATTCTCGTGCCTC | (SEQ ID NO:6) |
| CTCCGTGCTCTTAGTGAACTTGGGT | (SEQ ID NO:24) |
| TGTCAGCCAATCCTCCAGTTCGTAC | (SEQ ID NO:7) |
| CATGCTTGACCTCCTAACCGACTGT | (SEQ ID NO:25) |
| TTGTACGCCCTCCAAGAGAATCCTG | (SEQ ID NO:8) |
| GTCCTAAGAGAACCTCCCGCATGTT | (SEQ ID NO:26) |
| GCTCAAGCAATCCACCCGCCTCAG | (SEQ ID NO:9) |
| GACTCCGCCCACCTAACGAACTCG | (SEQ ID NO:27) |
| GGGATTACAGGCATGAGCCACCGC | (SEQ ID NO:10) |
| CGCCACCGAGTACGGACATTAGGG | (SEQ ID NO:28) |
| CACTCCATTTTTCTCACGGGCTGCA | (SEQ ID NO:11) |
| ACGTCGGGCACTCTTTTTACCTCAC | (SEQ ID NO:29) |
| CATTCTCCTGAGCCGTGATGCGAA | (SEQ ID NO:12) |
| AAGCGTAGTGCCGAGTCCTCTTAC | (SEQ ID NO:30) |
| ACGGAACGGGGTGTGAGCCTTGT | (SEQ ID NO:13) |
| TGTTCCGAGTGTGGGGCAAGGCA | (SEQ ID NO:31) |
| TGTCAGCTTGCTCTCACGGAACGG | (SEQ ID NO:14) |
| GGCAAGGCACTCTCGTTCGACTGT | (SEQ ID NO:32) |
| GGAGCTTATGCCTGGTGAGATCGTG | (SEQ ID NO:15) |
| GTGCTAGAGTGGTCCGTATTCGAGG | (SEQ ID NO:33) |
| GAGTCAGCAAGGAAGAGAAACGCG | (SEQ ID NO:16) |
| GCGCAAAGAGAAGGAACGACTGAG | (SEQ ID NO:34) |
| TGGATAAATTGCCTGGAATCAGCGC | (SEQ ID NO:17) |
| CGCGACTAAGGTCCGTTAAATAGGT | (SEQ ID NO:35) |
| CGTTGGATCTTGATAGCGAGACCGG | (SEQ ID NO:18) |
| GGCCAGAGCGATAGTTCTAGGTTGC | (SEQ ID NO:36) |

### Example 2

### Transfection of Antisense Oligonucleotides into Colon Cancer Cell Line

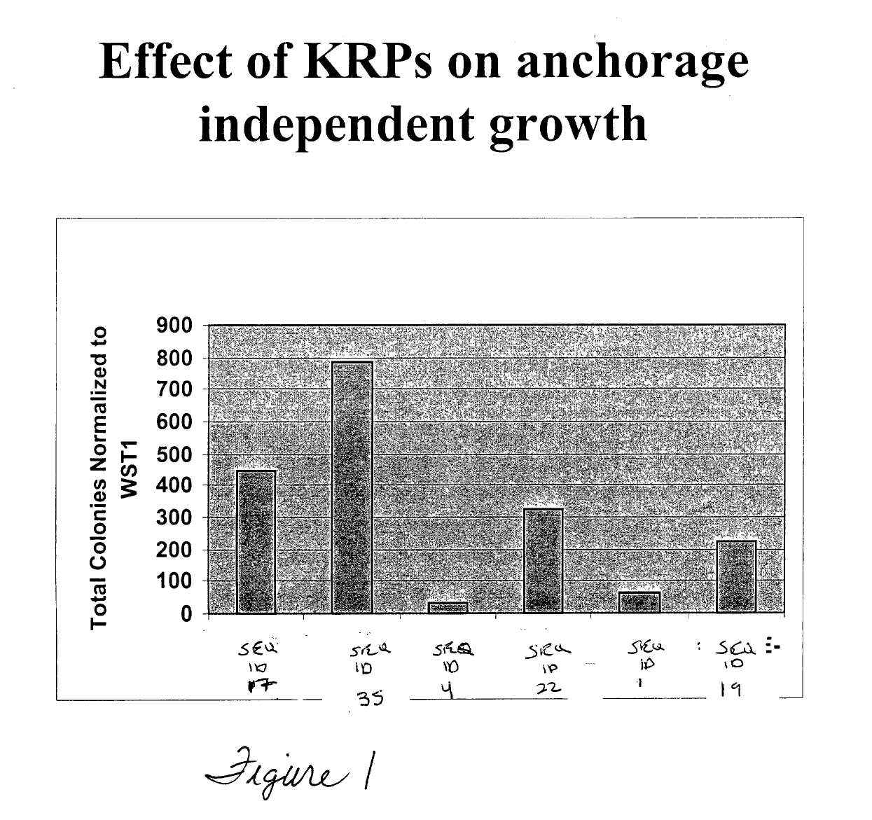

The antisense oligonucleotides (SEQ IDNO:17), (SEQ IDNO:4), and (SEQ ID NO:1) were each transfected into the human colon cancer cell line SW620. Additionally, the same colon cancer cell line was transfected with the corresponding reverse control sequences (respectively SEQ ID NO:35, SEQ ID NO:22 and SEQ ID NO:19). It was observed that the antisense oligonucleotides significantly inhibited the capability of the cells to grow in soft agar. These results indicate that these antisense oligonucleotides inhibited anchorage independent growth. These assay results contained in FIG. 1 indicate that the synthesized kinesin antisense oligonucleotides inhibit tumorigenesis.

### Example 3

### Effect of Antisense Oligonucleotides on Cell Cycle Profile

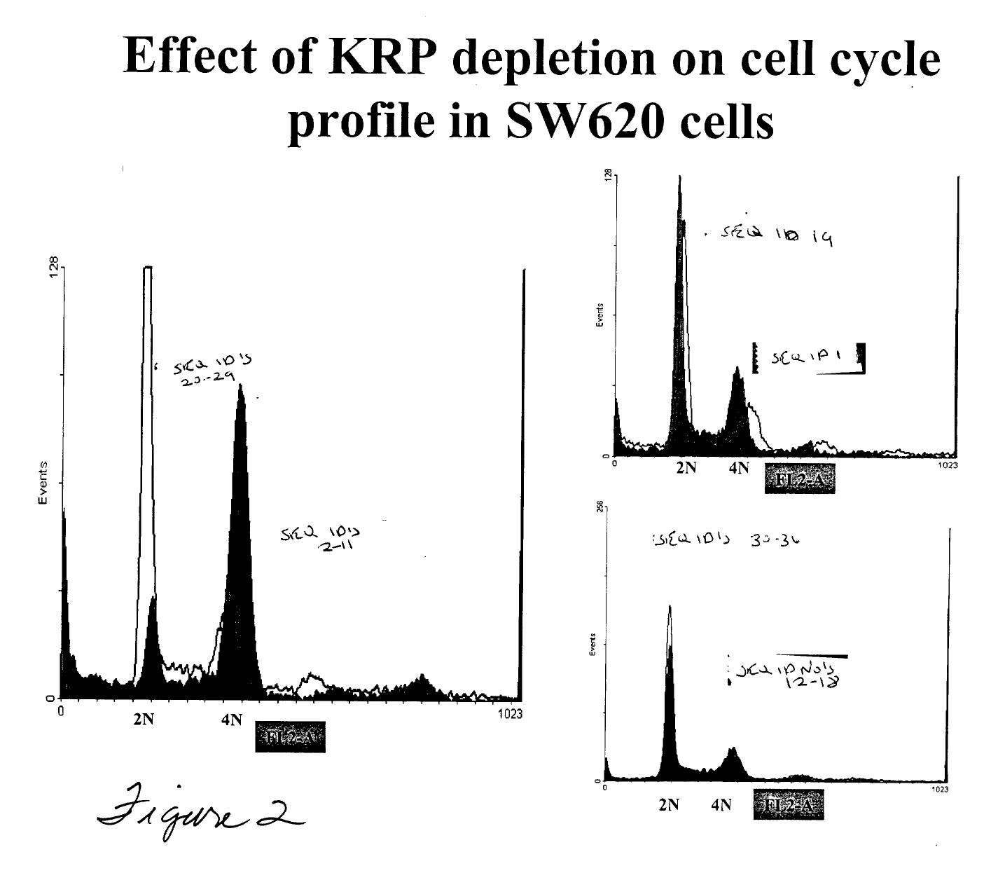

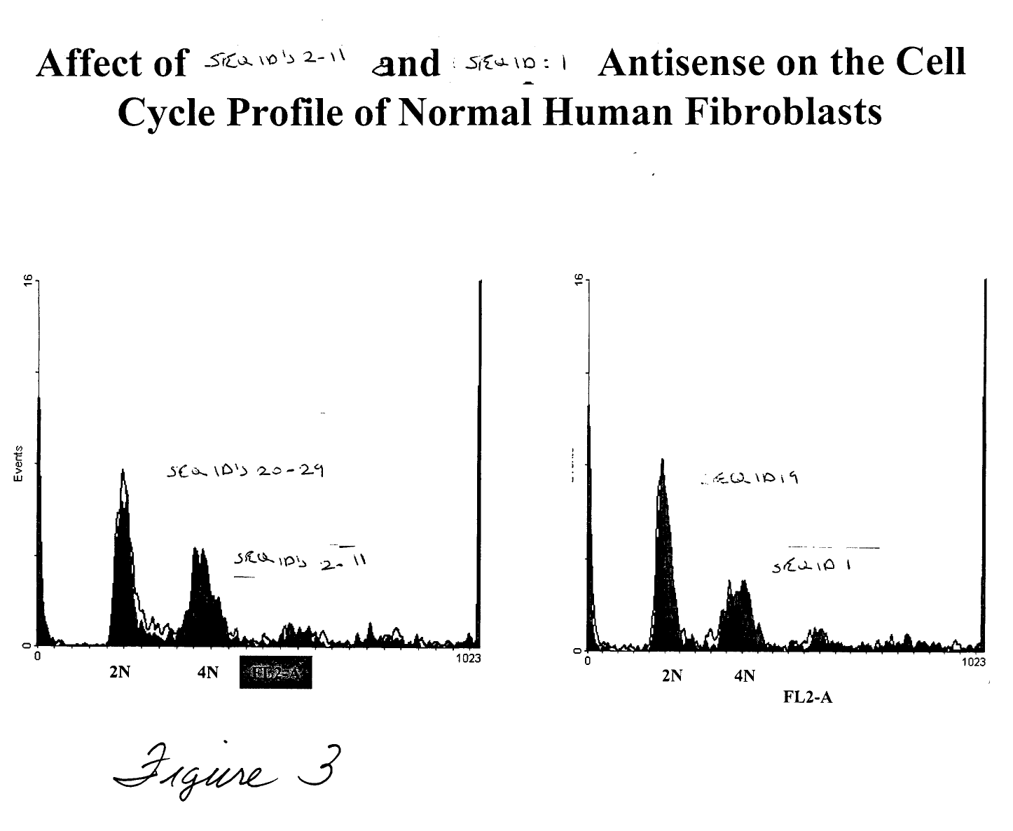

SW620 cells were transiently transfected with antisense oligonucleotides according to the invention and the effect on cell cycle profile evaluated by FACS analysis of propidium iodide stained cells. It was observed that cell tested antisense oligonucleotides arrested cells in G2/M phase to different degrees. Of the tested antisense oligonucleotides, the antisense oligos identified as SEQ ID NOs:2-11 had the most inhibiting effect, with SEQ ID NO:1 having the next best inhibiting effect and the antisense oligos identified as SEQ ID NOs:12-18 having the least inhibiting effect on cell cycle arrest. These results are contained in FIG. 2. Also, the effect of the same antisense oligonucleotides on the cell cycle of normal human filonblasts was tested. These results contained in FIG. 3 indicate that antisense oligos according to the invention affect normal and tumorigenic cells differently.

### Example 4

### Effect of Antisense Oligonucleotides on Cisplatinum Cytotoxicity

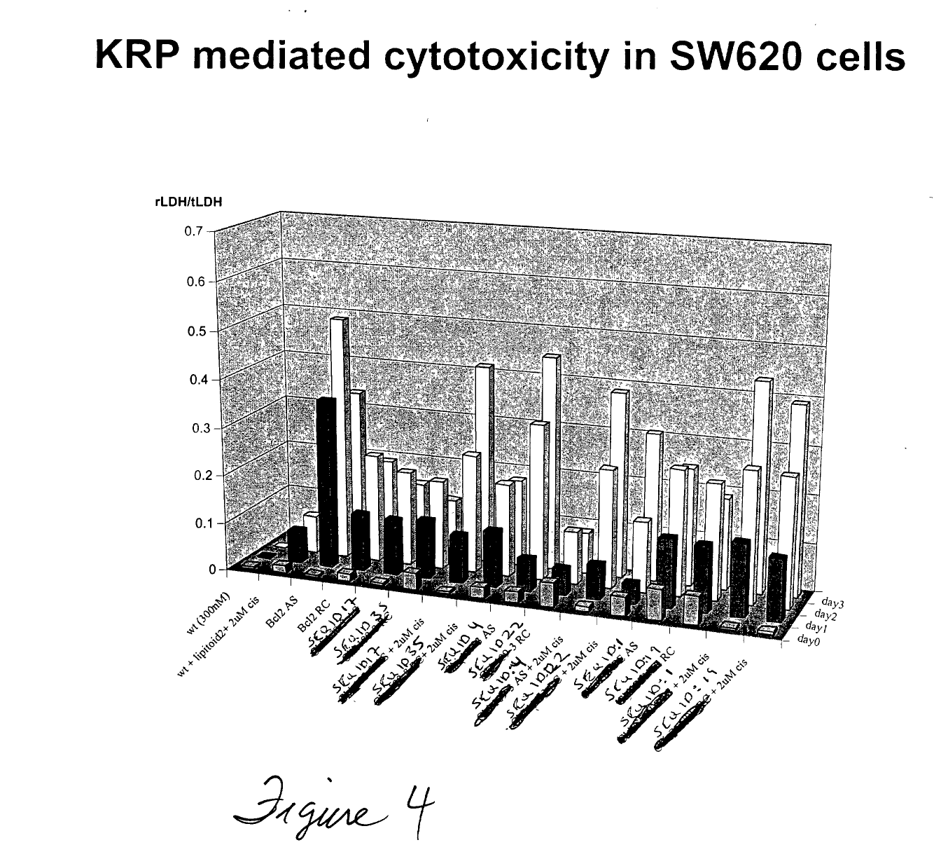

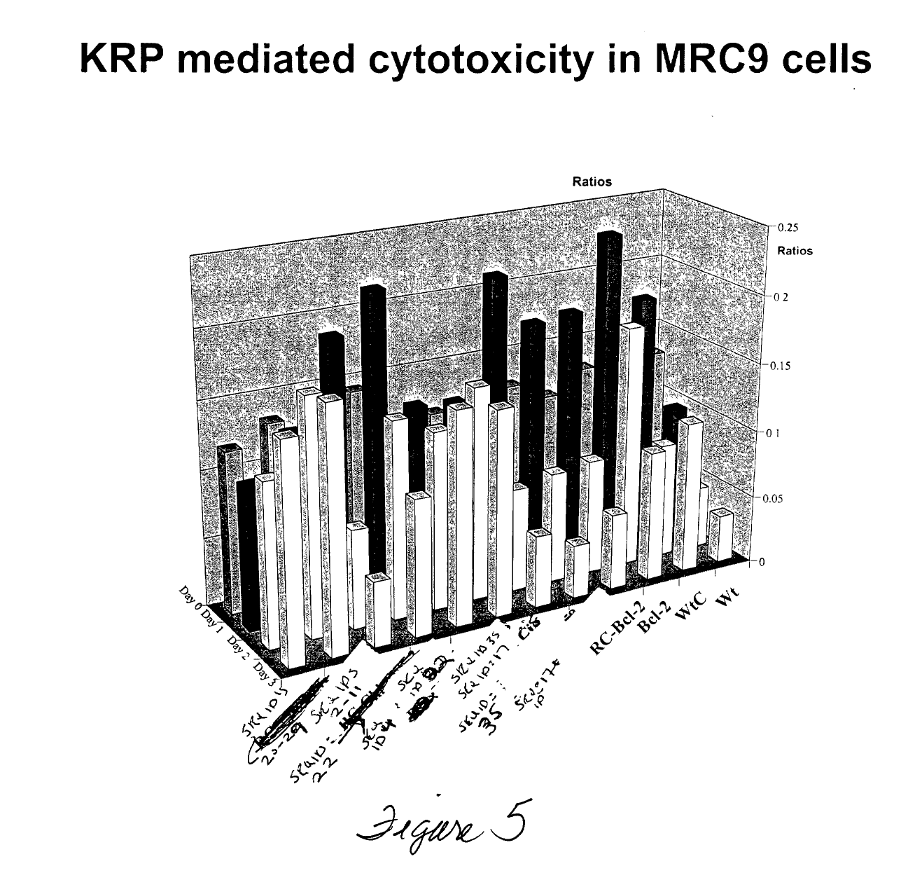

LDH cytotoxicity assays were performed to determine the effect of antisense oligonucleotides on SW620 cell death alone and in the presence of the chemotherapeutic drug cisplatinum. These results are contained in FIG. 4. It was found that the SEQ ID NO:4 oligonucleotide alone induced death of HT1080 cells. The SEQ ID NO:17 antisense oligonucleotide showed an increase in cytotoxicity when used in combination with the chemotherapeutic drug cisplatinum. These results suggest that antisense oligonucleotides according to the invention may be used to enhance the cytotoxic efficacy of this drug during chemotherapy, thereby reducing the requisite dose for chemotherapy. By contrast, the SEQ ID NO:1 antisense oligonucleotide did not induce cytotoxicity under the same conditions. Also, the same LDH cytotoxicity assays were performed on MRC9 cells transfected with the same oligonucleotides in the presence or absence of cisplatinum. These results are contained in FIG. 5. It can be seen upon comparison of the results in FIGS. 4 and 5 that the antisense oligonucleotides mediated cytotoxicity of SW620 cells did not yield the same results in MRC9 cells.

### Example 5

### Effects of Kinesin Antisense Oligonucleotides on Cell Proliferation

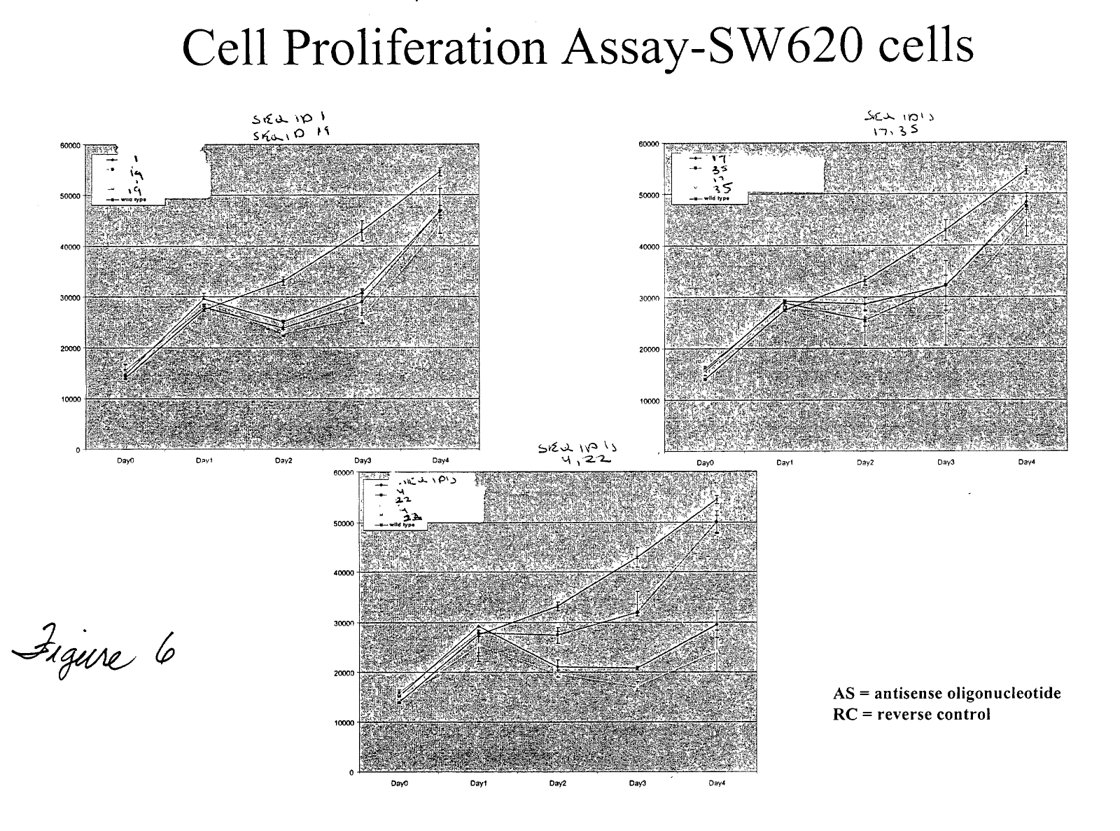

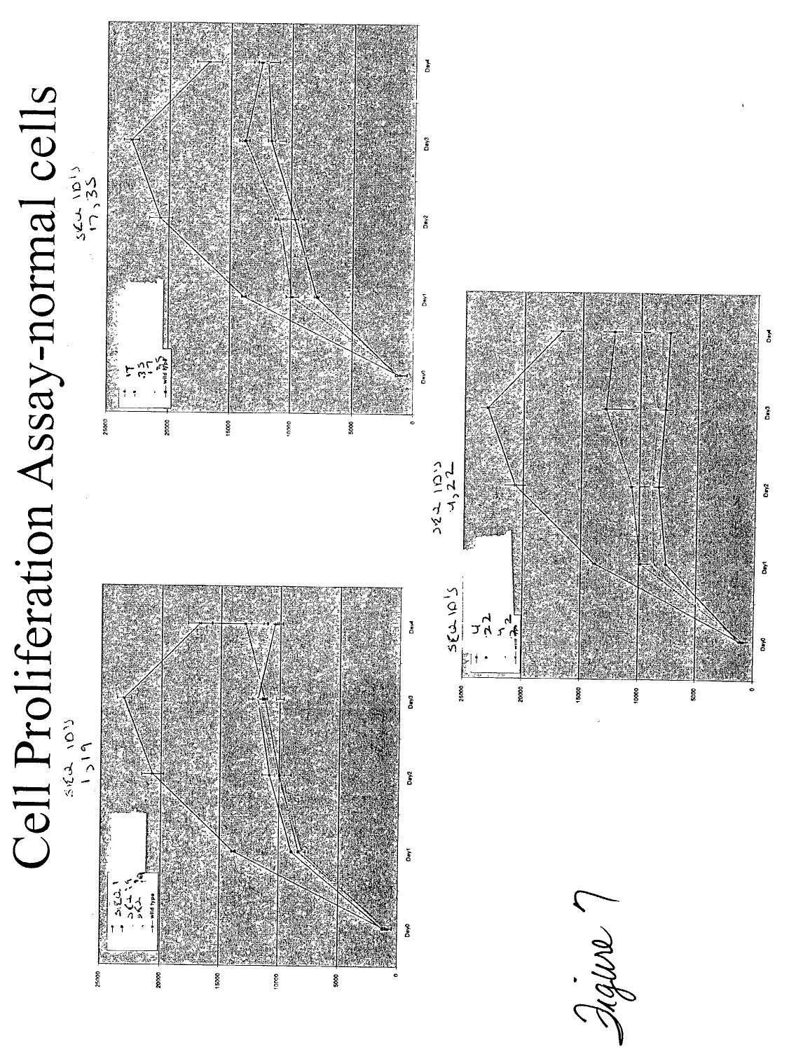

Experiments were further conducted to assay the effects of several kinesin anti-sense oligonucleotides (SEQ ID NO:1, SEQ ID NOs:12-18, AND SEQ ID NOs:2-11) and the corresponding reverse controls on the proliferation of SW620 cells and normal cells over a period of four days. These results are respectively contained in FIGS. 6 and 7. The results contained therein provide further evidence that kinesin antisense oligonucleotides inhibit proliferation of tumor cells.

### Example 6

### Treatment of Colon Cancer Patient with Antisense Oligonucleotides According to the Invention

A phosphate buffered saline composition containing an antisense oligonucleotide as described in Example 1 is produced and is administered to a patient with colon cancer by subcutaneous injection. After injection, the status of the tumor is monitored by MRI. Treatment is preferably continued until tumor ablation is achieved.

### Example 7

### Treatment of Colon Cancer by Combination Antisense/Chemotherapy

A phosphate buffered saline composition containing an antisense oligonucleotide according to Example 1 is prepared and administered to a colon cancer patient by subcutaneous injection. Within about one to seven days of antisense treatment, the patient is administered cisplatinum. After treatment, the status of the patient is monitored by MRI. Treatment is preferably maintained until tumor ablation is achieved.

What is claimed:

1. A method for treatment and/or prophylaxis of a disease involving aberrant cell proliferation comprising administering a therapeutically or prophylactically effective amount of an antisense oligonucleotide that inhibits or reduces the expression of a human kinesin gene selected from the group consisting of CENP-E, human Eg5 and MCAK.

2. The method of claim 1 wherein CENP-E gene has the nucleic acid sequence deposited in GenBank as GenBank ID Z15005.

3. The method of claim 1 wherein said human Eg5 gene has the nucleic acid sequence deposited in GenBank as GenBank ID U37426.

4. The method of claim 1 wherein said human MCAK gene has the nucleic acid sequence deposited in GenBank as GenBank ID U63743.

5. The method of claim 1 wherein said anti-sense oligonucleotide comprises a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or a portion thereof that inhibits or reduces expression of said human kinesin gene.

6. The method of claim 1 which is used for the treatment and/or prophylaxis of cancer.

7. The method of claim 6 wherein said cancer is selected from the group consisting of colon cancer, T and B cell lymphomas, pancreatic cancer, breast cancer, leukemia, bladder cancer, stomach cancer, brain cancer, esophageal cancer, liver cancer, adrenalcarcinoma, lung cancer, testicular cancer, heart cancer, ovarian cancer, uterine cancer, head and neck cancer, bone cancer, cervical cancer, gall bladder cancer, parathrnoid cancer, penile cancer, prostate cancer, skin cancer, spleen cancer, thymus cancer, thyroid cancer, muscle cancer, ganglial cancer, melanoma, myeloma, sarcoma and teratocarcinomas.

8. The method of claim 6 wherein said cancer is a digestive cancer or a lymphoma.

9. The method of claim 8 wherein said digestive cancer is selected for the group consisting of colon cancer, stomach cancer, pancreatic cancer, liver cancer, gall bladder cancer and esophageal cancer.

10. The method of claim 1 wherein said disease is selected from the group consisting of an autoimmune disorder, viral infection, neurological disorder, and condition associated with ischemia.

11. The method of claim 1 wherein said disease is a liver or pancreatic disease.

12. A combination therapy for treatment and/or prophylaxis of a disease condition which involves aberrant cell proliferation that comprises the administration of at least one chemotherapeutic or radionuclide and further comprises the administration of at least one anti-sense oligonucleotide that inhibits the expression of a human kinesin gene selected from the group consisting of CENP-E, human Eg5 and MCAK, wherein said chemotherapeutic or radionucleotide, and said at least one anti-sense oligonucleotide may be administered separately or in combination, and in either order, wherein said anti-sense oligonucleotide enhances.

13. The method of claim 12 wherein said CENP-E gene has the nucleic acid sequence deposited in GenBank as GenBank ID Z15005.

14. The method of claim 12 wherein said human Eg5 gene has the nucleic acid sequence deposited in GenBank as GenBank ID U37426.

15. The method of claim 12 wherein said human MCAK gene has the nucleic acid sequence deposited in GenBank as GenBank ID U63743.

16. The method of claim 12 wherein said anti-sense oligonucleotide comprises a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or a portion thereof that inhibits or reduces expression of said human kinesin gene.

17. The method of claim 12 wherein said chemotherapeutic is cisplatin.

18. An anti-sense oligonucleotide that inhibits expression of a human kinesin gene that is selected from the group consisting of a nucleic acid sequence having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or a portion thereof that inhibits or reduces expression of said human kinesin gene selected from the group consisting of CENP-E, human Eg5 and MCAK.

19. A pharmaceutical composition comprising at least one anti-sense oligonucleotide according to claim 18 and a pharmaceutically acceptable carrier.

20. The pharmaceutical composition of claim 19 which is suitable for administration by a route selected from the group consisting of oral, injection, intranasal, anal and topical administration.
